SEQUENCE LISTING (4 sequences)

SEQ ID NO: 1 - DNA, 1694 bp, Saccharomyces cerevisiae
  misc_feature: PromBNA2
gttgactctttcttagaatatgaaaaaaaaaaggggctccggtggtgatgaggagagaatatatatgcatgtatcaatcaagaccatttctaatgaccaccaccaccaatttcaatggttggtgggcaacccaaagtgctctttaaagaaccataaataccaaattgcaaggaggtcaaggtaccaaccatgaccaaacgagtaggcaacccagcaaaggaaccgaaaaatcctaactgtttggccaactgagccaacaaaccgactgtggattggccaggagccttcttagtcttgttgaccttggacaacagagtgtctgctggctgggaaacaatggcagcggccaaaccagcagtcaaaccggaaagcaaattcaacaaagtagtggaagtagaggacaatttttcttttggaccggcgaacccgtagtagaactcggaggcacgttcaaagaccaagaatttagcaatgttgtaaggaatttgcttgaacaaaattggagtgaacccactgtagaaagagccgataccttcttctttcaaaattctggaaaaccctccgacgagtccattagcgaactgtggttgggagaccaatctgattctagtggcttctaggggacacaaggcaatgtctgccaagaattcggccatggcagcagaacccatgtaaacagagttcttgtagcgagaggcggtatcgtaacccaagttgtcaatgaagaacttcttgaacacttcgtaaccaccgaacttgaaggcaccttggatagagtaacccaacaaagtaggaccaaacccggtcaacaacgcaccagcaccttctccggcgatgatttgcttgaaagagccgaccatacccttgttgtacacggtaggttccagttggattctcgtcttaacgacatcgatcgggaccatactggagtgagtcgacccgcaccctatggcgccggctagggcgaatttcatgtagtcagagacagagtactgtggaatagcaggagcagcagacacagacatggtgagatgagactttttgatctttgtagttcttcttcttgcagatcactctgttctttgcgatggttcttgtagataaaaagatcgggaggatggatcgcacttaaaaatcttctcgaaacgggccgcggccgggcatgggacccgttctcgacgctgattggctaacgatggttgggcagagtctcagctgctgcttgctcacattcgacaaaagactagaggaaagcagtaccatgctgcgatactgttgcatcgcagcattccgctgtccaaaaacaatacctgttgtattgtttgaggctggcaggcgcttgggtagaaacaacacccgatactggtgacagggctatctataaatattaaaacttgactccgcaattcgtcgtaaactgccttaaccatcgtaatggacgccgtgcaaatgcttcgacttttcccagtagtcaggcattgcgccagacacttgtgctgcttaattgtcgtaatgaaaacatatcggtagcggcctttttttattttttttctctatgggctgacgctgtcacttttcaacgtcgctccgaggcaaaaaagccaccgtcagaaataattactgtcacacacactagatgtccatgtatctttagcatatataaagcgaaaataaaagaaagagtcaacgccgat

SEQ ID NO: 2 - DNA, 200 bp, Saccharomyces cerevisiae
  misc_feature: TermBNA2
cattatacattttattaacgcccccccttttttttttttttttgttgatgcagaagcctcgcaacttaaagcttttcagttgtttcttcttggtcatattttttatacatatatacatacatatatgcatgtgctgtgtactacatgctgcctttcttcaatttttatgtatagacaaggaggccagaaagatattataa

SEQ ID NO: 3 - DNA, 560 bp, Saccharomyces cerevisiae
  misc_feature: PromNPT1
ttctacactctacagtttgaatacactttaactatcatcccattgtgattttattcaatgtttctttttttttttcttatgaaattttttttaccaaatacaacgagtgagaaattacttttcgttcaacgtgatctttgtgttctcaattgctgacacttccagtgtagtagccatcggagtgctgatagtagcttattttgggcttaaaaacctcaagaatatgaaaagatttatcaggtctgcttttgcatatatgaacccttcatcttacttttttacgttgtaaattcgtcaaaatatcaacaaaaatggctgctgcgcctgggtgaaaaattcgtcagatccgttcttataagactggaaaaactcgcaaatgaaagaattgttgtctttgactaaccaaaatgacgagcctttcaacagttatcgaaacgtaaattggatcccatcagtttttttgtttcaattcgtcactgtaaggaaacaaacggtagataatattttagaagaaaggtggatttagccttttgtacctattttcgccaagcaagataacc

SEQ ID NO: 4 - DNA, 200 bp, Saccharomyces cerevisiae
  misc_feature: TermNPT1
ttattcagttatgggtactgacatctctgcgattttataaatatgacatatagatatgcagctgatatcgcaaaaataaaacattatatgaagttgcgagtacgtaaaaaaacccgtgtttctttatgaggagattgttaattgtagatttatttttttttatttttaatgtatctatataatgaaataaaatgcaacga

(19) United States
(12) Patent Application Publication
(10) Pub. No.: US 2010/0081699 A1
(43) Pub. Date: Apr. 1, 2010

(54) YEAST PLATFORM CONSTRUCTION AND SCREENING METHODS

(75) Inventors: Christophe Francois Aimé Roca, Lisboa (PT); José Manuel Bernardo Sousa, Lisboa (PT); Marta Isabel Heitor Cerejo, Lisboa (PT); Alexandra Maria Barros Dos Santos, Lisboa (PT); Cátia Santana Reverendo Rodrigues, Lisboa (PT); Ricardo Filipe Antunes Pinheiro, Lisboa (PT); Johannes Sam, Freising (DE); Patricia Ramalhete Mendes da Silva Calado, Lisboa (PT); Sukalyan Chatterjee, Lisboa (PT); Helena Margarida Moreira de Oliveira Vieira, Lisboa (PT)

Correspondence Address: Joseph R. Baker, APC;Gavrilovich, Dodd & Lindsey LLP, 4660 La Jolla Village Drive, Suite 750, San Diego, CA, 92122, US

(73) Assignee: Bioalvo-Servicos ,Investigacao e Desenvolvimento em Biotlecnologia S.A., Lisboa (PT)

(21) Appl. No.: 12/520,348

(22) PCT Filed: Dec. 19, 2007
(86) PCT No.: PCT/PT2007/000051

§ 371 Date: Nov. 9, 2009

(30) Foreign Application Priority Data
Dec. 19, 2006 (GB) 0625310.8

(51) Int. Cl. A61K 31/403; C12N 5/00; C12N 1/19; C12N 5/071; C12N 5/07; C12Q 1/02; C12Q 1/68; C07D 209/82; A61P 3/00

(52) U.S. Cl. 514/411; 435/325; 435/254.2; 435/366; 435/354; 435/29; 435/6; 548/444

(57) ABSTRACT

The present invention relates to a cell which is suitable for screening a candidate agent as being an inhibitor of the metabolism of tryptophan to NAD+ and/or a modulator of NAD+ levels, which cell comprises functional genes of a pathway enabling the metabolism of tryptophan to NAD+ and wherein the cell includes a copy of an exogenous gene of said pathway, from the same or different species as the cell, which exogenous gene is under the control of an inducible or constitutive promoter and wherein any endogenous copy of the gene having the same function as the exogenous gene is a non functioning gene. The present invention also relates to populations of such cells and to methods of screening candidate agents with such cells.

The present invention relates to a cell which is suitable for screening a candidate agent as being an inhibitor of the metabolism of tryptophan to NAD⁺ and/or a modulator of NAD⁺ levels, which cell comprises functional genes of a pathway enabling the metabolism of tryptophan to NAD⁺ and wherein the cell includes a copy of an exogenous gene of said pathway, from the same or different species as the cell, which exogenous gene is under the control of an inducible or constitutive promoter and wherein any endogenous copy of the gene having the same function as the exogenous gene is a non-functioning gene. The present invention also relates to populations of such cells and to methods of screening candidate agents with such cells.

Indoleamine 2,3-dioxygenase (IDO; MW 48,000; EC 1.13.11.42) and tryptophan 2,3-dioxygenase (TDO) are heme-containing enzymes that are the first and rate-limiting enzymes in mammalian tryptophan metabolism. Both enzymes catalyze the oxidation of the essential amino acid tryptophan to N-formylkynurenine by dioxygen and are responsible for processing tryptophan in the human body. Although both dioxygenases catalyse the same reaction, they are distinct in several respects of their molecular and immunogenic properties, substrate specificities, biological sources, and tissue distribution. TDO is expressed constitutively in the liver in contrast to IDO, whose expression and activity in extrahepatic tissues is under the regulation of the immune response. While TDO has two binding sites for L-Trp (a catalytic and regulatory site) L-Trp does not exert a regulatory action upon IDO, but rather exhibits substrate inhibition. However, there is strong evidence for an effectors binding site in IDO near the substrate binding site and the heme iron which enhances substrate affinity. Indole derivatives with substituents at the 3-position have been reported to serve as effectors in vitro, although the natural biological effector is unknown. Furthermore, IDO is known to be inhibited in a non-specific manner by general inhibitors of heme-containing enzymes. Also, certain tryptophan (substrate) analogues such as 1-methyl-L-tryptophan (1-MT) and beta-(3-benzofuranyl)-DL-alanine are competitive inhibitors of IDO.

Infection with certain viruses, bacteria and parasites induces an immune response mediated by lymphocytes that release IFNα. This cytokine is a potent inducer of IDO in infected host cells. Consequently, tryptophan, an essential amino acid for pathogens, is degraded and thus infection prevented. Moreover, one of the terminal tryptophan catabolites, picolinic acid, can activate and enhance the microbiocidal response of the host cell. Besides playing a role in control of infection, IDO is immunosuppressive when expressed by tumors. The T-cell mediated rejection of the tumor is prevented because IDO activity induces tryptophan depletion in the extracellular medium blocking T-lymphocyte cell progression in the cell cycle. Likewise, the IDO activity in antigen presenting cells (APCs) that interact with T cells inhibits T cell responses. In this case, the kynurenines, some of them as proapoptotic molecules, regulate T cell survival. The immunosuppressor function of IDO is further demonstrated during pregnancy where the IDO expression in the placenta is crucial to prevent an immune response of the maternal T cells to fetal tissues that express alloantigens.

In the Central Nervous System (CNS), IDO induction by certain infections or neurological disorders depletes the available free tryptophan pool reducing the serotonin production and increasing the concentration of several tryptophan catabolites that are neuroreactive. Catabolites such as quinolinic acid, an agonist of the NMDA receptors, and 3-Hydroxykynurenine are neurotoxic and are involved in the pathology of the CNS. Kynurinic acid is anticonvulsant and has neuroprotective effect. This combination contributes to the development of many neurological/psychiatric disorders and is a factor in several mood disorders as well as related symptoms in chronic diseases characterised by IDO activation and tryptophan degradation, such as acquired immune deficiency syndrome (AIDS), Alzheimer's disease, several types of depression and cancer.

Tryptophan metabolism is also intrinsically connected to NAD⁺/NADH levels in the cell and it is well known by the skilled person that perturbations on this homeostasis level conducts to age related diseases, such as neurodegenerative disorders, cancer or type 1 and 2 diabetes. The NAD⁺/NADH redox state regulates the co-repressor CtBP activity and therefore plays a role in carcinogenesis. In addition, it is also thought possible that NAD⁺/NADH can regulate the tumour suppressor p53 via Sir2p. In type 1 diabetes, NAD levels are involved by depletion of its levels in β-cells via PARP1 and in type 2 this occurs via point mutations in the ND1 gene (NADH dehydrogenase) found in these patients. Since this is a dysfunction at the mitochondrial complex I it is also possible that NAD⁺ levels are crucial for the development of other diseases involved with this system, such as Parkinson's disorder.

It is well recognized in the art, that although some modulators of IDO activity are available, potent inhibitors with potential therapeutic use are still not available. Such means would provide for treatment or prophylaxis of disease conditions which result from the products of tryptophan degradation, and/or in appropriate levels of NAD⁺/NADH, such as viral infections including AIDS, bacterial infections, neurodegenerative disorders (e.g. Alzheimer's, Huntington's and Parkinson's diseases), depression, cancer, conditions of the eye and autoimmune disorders. As currently practiced in the art, the drug discovery process to identify suitable therapeutic agents is a long and multiple step process involving identification of specific disease targets, development of an assay based on a specific target, validation of the assay, optimization and automation of the assay to produce a screen, high throughput screening of compound libraries using the assay to identify “hits”, hit validation and hit compound optimization. The output of this process is a lead compound that goes into pre-clinical trials and, if validated, eventually into clinical trials. In this process, the screening phase is distinct from the assay development phases, and involves testing compound efficacy in living biological systems.

Therefore, there is a need in the art of a system where one can identify more easily those therapeutic agents needed for the diseases stated above which may have their therapeutic effect due to being an inhibitor of the metabolism of tryptophan to NAD⁺and/or a modulator of NAD⁺/NADH levels. Furthermore there is a need that such a system provides a robust, efficient, rapid and cost-effective method to screen for compounds that will have a therapeutic effect in treatment or prophylaxis of disease conditions which result from the products of tryptophan degradation pathway and/or differing NAD⁺/NADH levels in the cell.

The present invention provides a yeast platform design/construction and screening methods for identifying substances that have a therapeutic value for various diseases associated with NAD⁺ levels and/or tryptophan catabolism. The platform and methods herein described provide a robust, efficient, rapid and cost-effective method to screen for compounds that will have a therapeutic effect in treatment or prophylaxis of disease conditions which result from the products of tryptophan degradation pathway, such as neuroinflammation, foetal-rejection, tumours, AIDS, cerebral malaria and many others, and of NAD⁺-associated diseases such as carcinogenesis, type 1 and 2 diabetes and neurodegenerative diseases such as Parkinson's.

Accordingly, a first aspect of the present invention provides a cell suitable for screening a candidate agent as being an inhibitor of the metabolism of tryptophan to NAD⁺ and/or a modulator of NAD⁺ levels, which cell comprises functional genes of a pathway enabling the metabolism of tryptophan to NAD⁺ and wherein the cell includes a copy of an exogenous gene of said pathway, from the same or different species as the cell, which exogenous gene is under the control of an inducible or constitutive promoter and wherein any endogenous copy of the gene having the same function as the exogenous gene is a non-functioning gene. In general, modulators of NAD⁺ levels will effect (inhibit) the metabolism of tryptophan to NAD⁺.

Cells most suitable for the first aspect of the invention are those which naturally contain functional genes of a pathway enabling the metabolism of tryptophan to NAD⁺ or have already been engineered to do so. Alternatively, the cell needs to be engineered to do so. Thus, typically most suitable cells for the first aspect of the invention are eukaryotic cells, such as yeast, human or mouse cells, although prokaryotic cells can be used. The yeast cell may be any strain of yeast, such as Saccharomyces cerevisiae or any other Saccharomycetates.

The cell according to the first aspect of the invention is useful for screening candidate agents for being inhibitors of the metabolism of tryptophan to NAD⁺ and/or modulators of NAD⁺ levels. The use of the cells of the invention, in screening, is to identify therapeutic agents which can be used to treat any disease or disorder which requires an inhibitor of the pathway of tryptophan to NAD⁺ or a modulator of NAD⁺/NADH levels for treatment. Such diseases and disorders include immune deficiency syndrome (AIDS), Alzheimer's disease, depression, schizophrenia, mood disorders, multiple sclerosis, stroke, cancer, neuroinflammation, foetal-rejection, tumours, malaria, including cerebral malaria, carcinogenesis, types 1 and 2 diabetes, neurodegenerative diseases such as Parkinson's disorder etc., teratogenesis, Huntington's disease, dykinesia, epilepsy, meningitis and seizures.

Any deregulation of the kynurenine pathway (i.e. deregulation without specific particular enzymes being involved), and to some extent NAD⁺ levels, can result in diseases such as those described in the preceding paragraph. In addition, the following diseases are specifically associated with deregulation of the following enzymes.

TABLE 1

| Mammalian Enzyme | Known related disease | Equivalent yeast gene |
|---|---|---|
| Indoleamine | Alzheimer's disease | BNA2 |
| 2,3-dioxigenase | Cerebral malaria | |
| Tryptophan | Alzheimer's disease | BNA2 |
| 2,3.dioxygenase | | |
| Formamidase | Teratogenesis | BNA3 |
| Kynurenine | Huntington's and | ARO8/ARO9 |
| amino-transferase | Alzheimer's disease | |
| Kynureninase | Parkinson's disease | BNA5 |
| Kynurenine | Huntington's disease, | BNA4 |
| 3-hydroxylase | malaria, dyskinesia | |
| Kynurenine | Huntington's disease, | BNA4 |
| 3-monooxydase | malaria, dyskinesia | |
| 3-hydroxyanthranilic | Epilepsy, Seizure | BNA1 |
| acid dioxygenase | | |
| Quinolinate | Parkinson's disease, | BNA6 |
| phosphoribosyl | AIDS, meningitis | |
| transferase | | |

According to the first aspect of the invention, the cell includes at least one copy of an exogenous gene of said tryptophan to NAD⁺ pathway. Said exogenous gene can be from the same species as is the cell or a different species. Thus, a yeast cell can be used according to the first aspect of the invention to screen for inhibitors or modulators of a human gene. In general in current society, human disease treatments are those which are current sought after most. Accordingly, the exogenous gene is preferably human (or at least encodes a human protein if it is a coding gene).

The exogenous gene is under the control of a promoter, which promoter can be either inducible or constitutive. Such promoters include any one of the GAL1, GAL10, ADH1 or P6K promoters or any constitutive or inducible tissue specific promoter or element. The cell is preferably designed such that the exogenous gene is the only functioning copy of that particular gene, i.e. any copy of the equivalent gene in the cell should be non-functioning. By non-functioning is meant that it does not function as its normal role to any level which interferes with determining the inhibition or modulation of the exogenous gene by a candidate agent. A non-functioning gene can be damaged, disturbed, detected, but does not function. In another respect, any equivalent gene to the exogenous gene is entirely deleted (also non-functioning) so that there is no copy of any equivalent gene present in the cell.

The exogenous gene is any gene in the pathway for which it is desired to screen a candidate agent for its ability to inhibit the pathway or modulate NAD⁺ levels. The exogenous gene may encode any one or more of the following: Indoleamine 2,3-dioxygenase, tryptophan 2,3-dioxygenase, formamidase, kynurenine amino-transferase, kynureninase, kynurenine 3-hydroxylase, kynurenine 3-monooxydase, 3-hydroxyanthranilic acid dioxygenase, quinolinate phosphoribosyl transferase, nicotinate phosphoriboxyl transferase, nicotinamide/nicotinic acid mononucleotide adenylyltransferase, nicotinamide/nicotinic acid mononucleotide adenylyltransferase, glutamine-dependent NAD synthase, NAD-dependent histone deacetylase or nicotinamidase.

The copy of the exogenous gene may be located anywhere in the cell, either on the genome or preferably, for ease of use and design, outside of the genome. Such a location may be on a plasmid, episomal or centromeric.

According to a first aspect of the invention, the cell preferably also comprises a reporter gene which is under the control of a promoter, which promoter is regulated directly or indirectly by the expression product (usually a protein) of the exogenous gene. Thus, the level of activity of the exogenous gene determines the level of expression of the reporter gene (high or low, depending on whether the activity of the reporter gene upregulates or downregulates the promoter—either is possible). The reporter gene can be any known in the art, such as fluorescence protein (e.g. EGFP), XFP, β-galactosidase, luciferase, other enzymes, immunological markers or any other selectable and screenable marker. The promoter can be any which is regulated by the expression product of the exogenous gene. Suitable promoters include the promoter of the BNA2 gene, any NAD⁺ level-sensitive promoter, sequences such as TNA1 or other BNA gene promoters (except BNA3) or a sequence with the same activity thereof.

Preferably, the promoter of the reporter gene is downregulated, directly or indirectly, by the expression product of the exogenous gene. In this way, high activity (usually transcription and translation) of the exogenous gene results in low expression of the reporter gene, meaning that any candidate agent screening which provides low expression of the reporter gene is not a useful candidate for inhibition. However, any high inhibition of the exogenous gene activity (again, usually protein expression) will result in a high expression of the reporter gene in a dose-dependent manner. Of course, total inhibition of the exogenous gene will be lethal to the cell.

In the first aspect of the present invention, it is preferably that the cell comprises the functional genes of a single pathway enabling the metabolism of tryptophan to NAD⁺. Additional pathways (such as the salvage pathway in yeast) can provide background noise to any screening, which can be a disadvantage. Alternatively, it is preferably if such a pathway exists, it is either completely or partially rendered inactive, by damage, disturbance or detection of genes. Preferably, any gene enabling the metabolism of NA to NANM is non-functioning.

In a preferred embodiment of the first aspect, the exogenous gene encodes IDO and the promoter of the reporter gene is the promoter of the BNA2 gene. However, other combinations include any BNA gene promoters (excluding BNA3) or TNA1 promoter, with any kynurenine pathway or NAD⁺ salvage pathway encoding sequence.

A second aspect of the invention provides a population of cells according to the first aspect of the invention. All details and preferred embodiments of the first aspect of the invention also relate to the second aspect.

A third aspect of the invention provides a method of screening a candidate agent for its inability to inhibit the metabolism of tryptophan to NAD⁺ and/or to modulate NAD⁺ levels, the method comprising contacting the candidate agent with a cell according to the first aspect of the invention or a population of cells according to the second aspect of the invention and determining the ability of the candidate agent to inhibit the metabolism of tryptophan to NAD⁺ and/or to modulate NAD⁺ levels. This method according to the third aspect of the invention allows the candidate agent to be easily screened for its ability to either inhibit the metabolism of tryptophan to NAD⁺ and/or to modulate NAD⁺ levels. The assay provides efficient screening platforms to enable easy and fast screening to identify useful therapeutic compounds. The candidate agent can be any candidate agent including small molecules, compounds, larger molecules, enzymes, compound analogues, etc. A particular benefit of the invention is to screen a candidate agent using a first aspect of the invention, wherein the cell, or population of cells, comprises a reporter gene under the control of a promoter, which promoter is downregulated, directly or indirectly, by the expression product of the exogenous gene. This screening allows the determination of the less toxic, but more potent dosage of each potential inhibitor. In accordance with the present invention, “contacting” the candidate agent with a cell, includes exposing, incubating, touching, associating, making accessible, the cell to the candidate agent.

All preferred embodiments of the first and second aspects of the invention, also apply to the third.

A fourth aspect of the invention relates to an agent which inhibits the metabolism of tryptophan to NAD⁺ and/or modulates NAD⁺ levels, obtained by a method according to the third aspect of the invention. All preferred embodiments of the first to third aspects of the invention, also apply to the fourth.

A fifth aspect of the invention relates to a method of treating a patient who suffers or is predisposed to suffer from a disease which requires inhibition of the metabolism of tryptophan to NAD⁺ and/or modulation of NAD⁺ levels for amelioration, the method comprising screening a candidate agent according to the third aspect of the invention, to identify an agent which inhibits the metabolism of tryptophan to NAD⁺ and/or modulates NAD⁺ levels and administering the agent to the patient. According to the present text, the term “treating” relates to any treatment or partial treatment or prevention, to any extent, of a patient who suffers from or is predisposed to suffer from any disease. Most preferably, the patient is in need of treatment. The method may include tests on the patient to determine whether the patient is one who suffers from or is predisposed to suffer from the disease.

A sixth aspect of the invention relates to an agent according to the fourth aspect of the invention for treating a disease which requires inhibition of the metabolism of tryptophan to NAD⁺ and/or modulation of NAD⁺ levels for amelioration.

A seventh aspect of the invention relates to the use of an agent according to the fourth aspect of the invention in the manufacture of a medicament for treating a disease which requires inhibition of the metabolism of tryptophan to NAD⁺ and/or modulation of NAD⁺ levels for amelioration.

According to the fourth, fifth or sixth aspect of the invention, the therapeutic agent may be obtained by a method according to the third aspect of the invention. All preferred embodiments of the first to fourth aspects of the invention also apply to the fifth and further embodiments.

Inhibitors of the pathway from tryptophan to NAD⁺ and/or NAD⁺/NADH level modulator compounds, to be used in treatment or prophylaxis of disease conditions which result from the products of tryptophan degradation and unbalanced NAD⁺ levels, are still not available, nor are fast and efficient compound-screening platforms that can provide a specific setup for such purpose. This invention fulfils part of these needs and it is a significant contributor to rapidly fulfilling the need for these therapeutic compounds.

The invention is based on a platform that is sensitive to NAD⁺ levels within the cell and on the fact that inhibition along the pathway interferes with such NAD⁺ levels. This invention concerns a method for in vitro screening of direct or indirect NAD⁺ synthesis inhibitors (by in vitro here is meant outside of the human or animal body, although the cells of the screen are living cells).

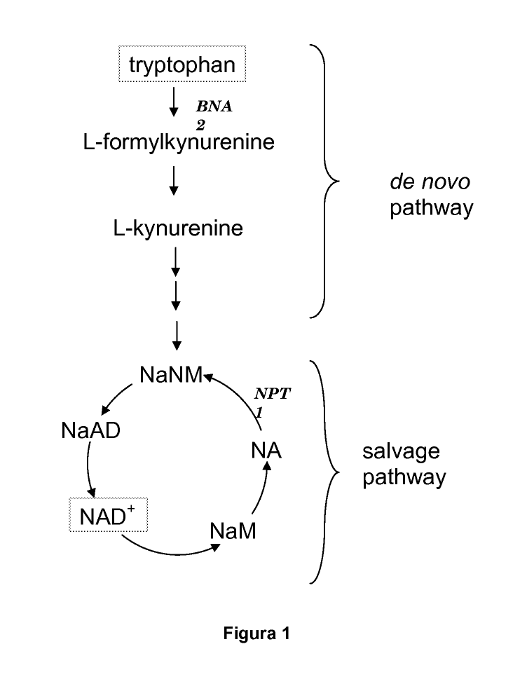

The invention can be exemplified by reference to the modification and the tight control of the NAD⁺ synthesis pathways in S. cerevisiae cells, depicted in FIG. 1.

Co-deletion of the two pathways in S. cerevisiae is lethal for the cell as it is not able to synthesize NAD⁺ (deletion of NPT1 causes synthetic lethality with deletion of genes in the kynurenine pathway). In the said invention, the lethality is overcome by the introduction of the human IDO gene under a strong inducible promoter (such as GAL1 promoter or any other constitutive/inducible, tissue specific promoter or element), and by the expression of the said IDO protein by growing the cells on galactose/specific inducing conditions. In the present embodiment, the complete inhibition of IDO by small molecules or any other bioactive compound would then result in cell death. The cell can only grow when IDO is active because no other pathway for NAD⁺ synthesis exists, resulting in a tight control of the effect of the inhibitors.

However, if the screening is only based on a life/death assay, pitfalls can be induced by the direct toxicity of the inhibitor on the cell, and therefore a potential action on the IDO activity would not be detected or detected as false positive (the cell will not grow because the inhibitor targets other essential pathways in the cell). In a further embodiment, in order to find the balance between the toxicity of the inhibitor and the minimal dosage for initiation of inhibition, a reporter gene (e.g. an enhanced green fluorescence protein (EGFP) gene) is cloned directly upstream the BNA2 ORF, resulting in the expression of the reporter gene under the induction of BNA2 promoter. In this way, the set-up additionally offers a rapid and visual assay for distinguishing true inhibition potential from deleterious toxicity. BNA2 is tightly regulated by the levels of NAD⁺ in the cell, via the repression of expression by binding of the Sum1p/Hst1p complex on the BNA2 promoter (Bedalov A, Hirao M, Posakony J, Nelson M, Simon JA. 2003. NAD⁺-dependent deacetylase Hst1p controls biosynthesis and cellular NAD⁺ levels in Saccharomyces cerevisiae. Mol Cell Biol. October; 23(19):7044-54).

Generally, when NAD⁺ levels are high (i.e. in the present preferred embodiment when IDO is active), the BNA2 promoter is repressed and no EGFP is expressed. When NAD⁺ levels are reduced (i.e. when the IDO is inhibited by small molecules), the Sum1p/Hst1p complex is released from the BNA2 promoter and the EGFP can be expressed (Table 3 in the examples). Thus, this screening allows the determination of the less toxic but more potent dosage of each potential inhibitor. In further embodiments, any enzyme whose activity is linked to NAD⁺ levels can be targeted with this set up as it controls the expression of the said reporter gene under the BNA2 promoter induction. Furthermore, the said reporter gene could be any fluorescent based marker, enzymes, immunological markers and any other example of selectable and screenable markers that are well known to one of skill in the art.

Such fast, efficient and conclusive high-throughput screening system for new molecules inhibiting NAD⁺ production, in particular through IDO inhibition has not previously been available. Thus, this platform system is now made available to facilitate the discovery of new IDO inhibitors giving a step forward to a real market need for compounds to be used in treatment or prophylaxis of disease conditions which result from the products of tryptophan degradation pathway.

The invention contemplates the use of the genetically modified cells as well as the inhibitor screening system described herein, including their use for other NAD⁺ modulators screening with biotechnological or pharmaceutical applications.

The present invention is described with references to the following figures, in which:

FIG. 1: NAD⁺ synthesis pathways in S. cerevisiae cells. NAD⁺ synthesis in S. cerevisiae. BNA2 encodes the first enzyme of the de novo (kynurenine) pathway, and NPT1 encodes the nicotinate phosphoribosyltransferase, responsible for the conversion of NA into NaMN. (Abbreviation: NaAD desamino-NAD⁺, NA: nicotinic acid, NaMN: NA mononucleotide; NaM: nicotinamide)

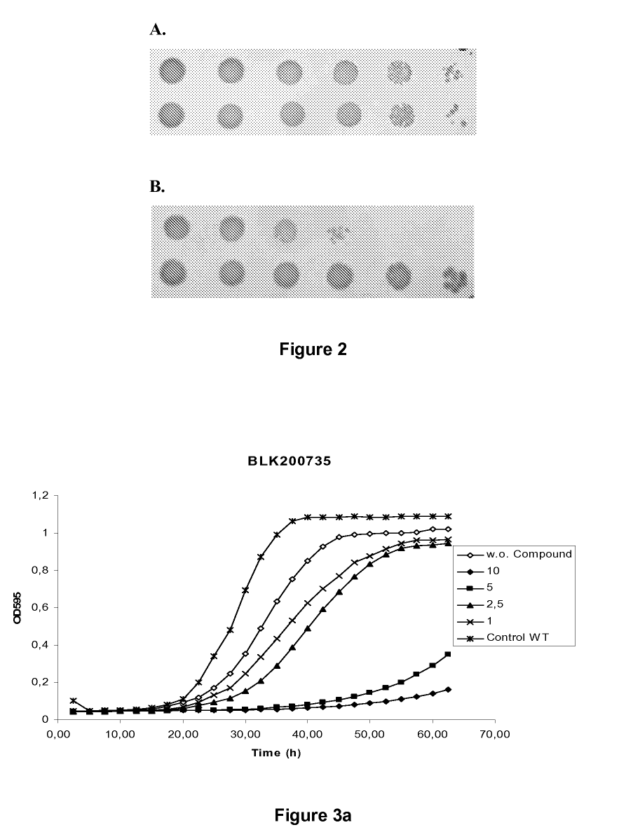

FIG. 2: YBlockade cells depend on the activity of human IDO to survive. Spotting on selective medium with induction (galactose-A) or non-induction (glucose-B). Upper line: BLOCKADE. Bottom line: wild type cells.

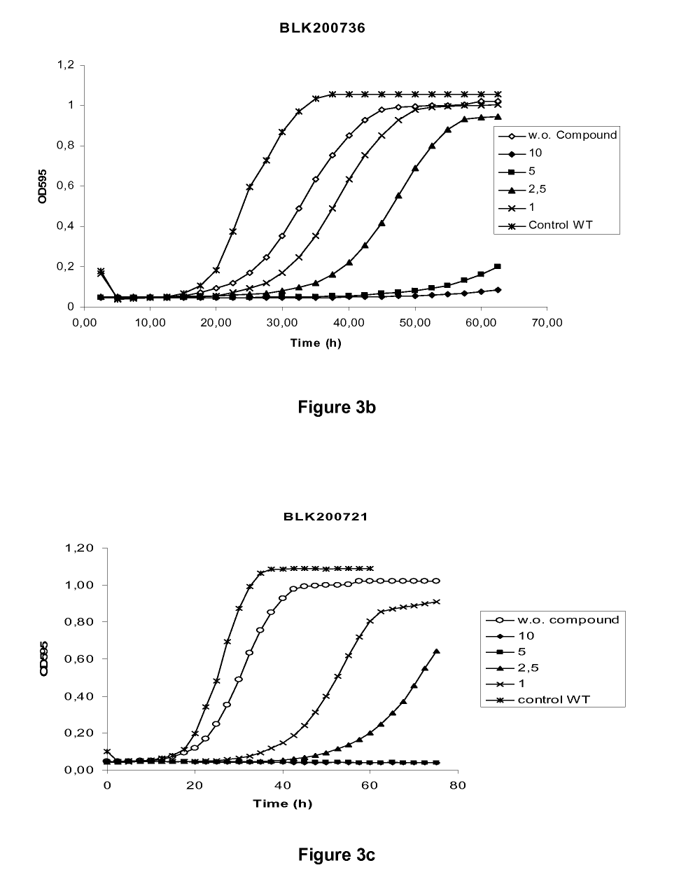

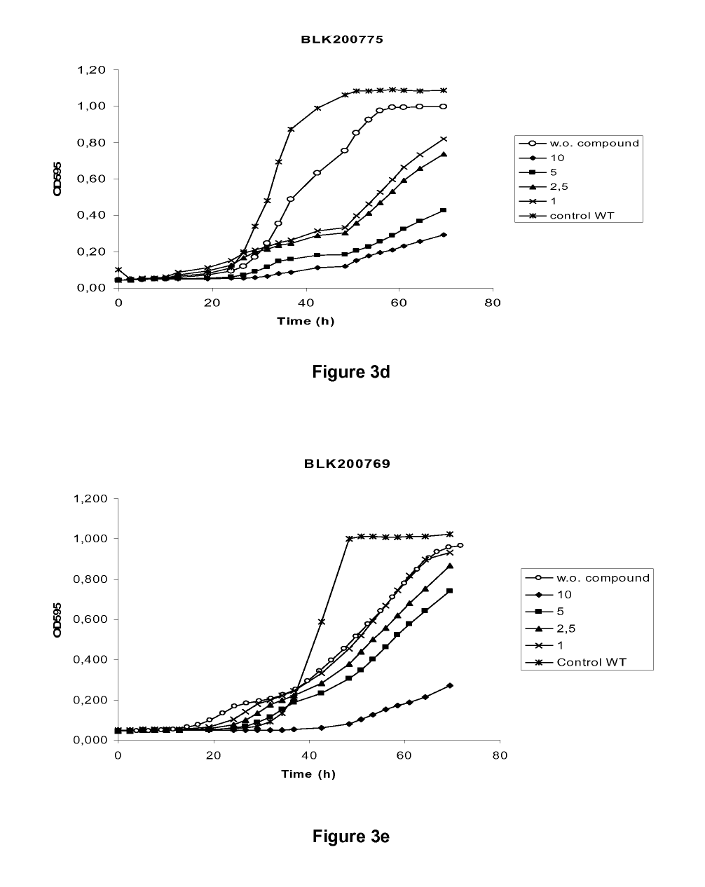

FIG. 3a: Growth of BLOCKADE cells on minimum medium with 2% galactose containing 10, 5, 2.5 and 1 μM of compound BLK200735. The WT cells were grown on minimum medium with 2% galactose containing 10 μM of compound BLK200735.

FIG. 3b: Growth of the BLOCKADE cells on minimum medium with 2% galactose without compound or containing 10, 5, 2.5 and 1 μM of compound BLK200736. The WT cells were grown on minimum medium with 2% galactose containing 10 μM of compound BLK200736.

FIG. 3c: Growth of the BLOCKADE cells on minimum medium with 2% galactose containing 10, 5, 2.5 and 1 μM of compound BLK200721. The WT cells were grown on minimum medium with 2% galactose containing 10 μM of compound BLK200721.

FIG. 3d: Growth of the BLOCKADE cells on minimum medium with 2% galactose containing 10, 5, 2.5 and 1 μM of compound BLK200775. The WT cells were grown on minimum medium with 2% galactose containing 10 μM of compound BLK200775.

FIG. 3e: Growth of the BLOCKADE cells on minimum medium with 2% galactose containing 10, 5, 2.5 and 1 μM of compound BLK200769. The WT cells were grown on minimum medium with 2% galactose containing 10 μM of compound BLK200769.

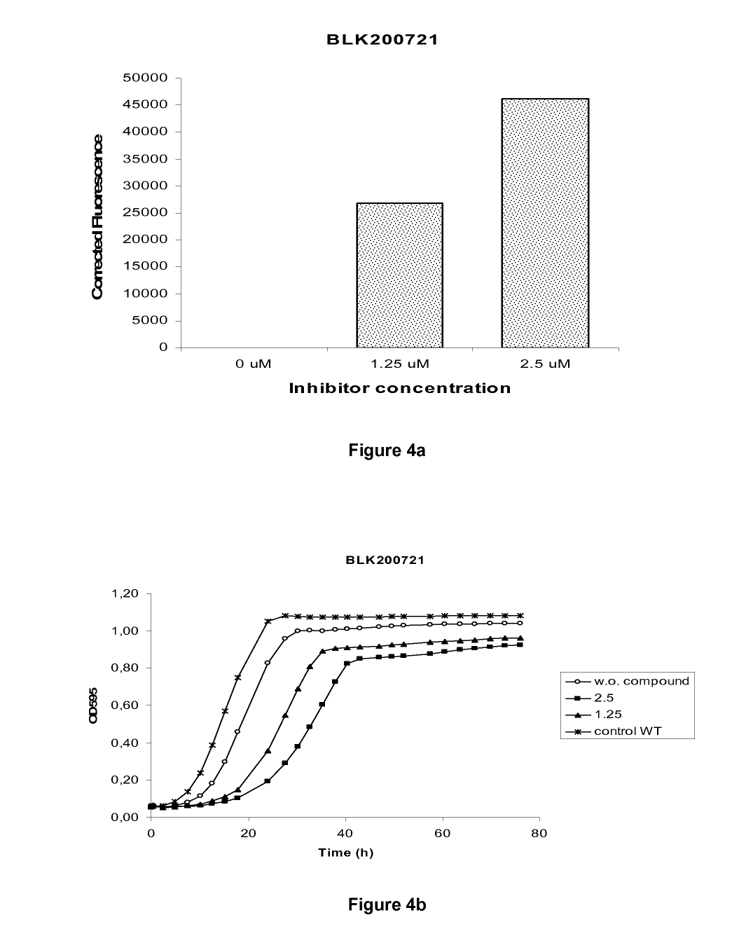

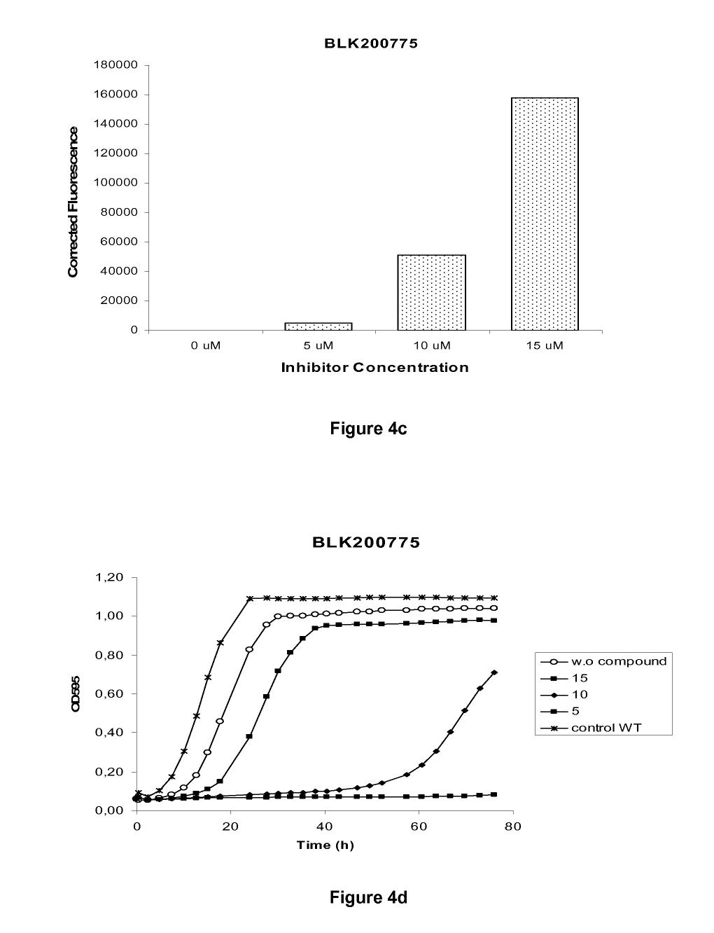

FIG. 4a: Representative example of fluorescence production when cells are submitted to increasing concentrations of inhibitor compound BLK200721. Higher concentrations than 2.5 μM result in cell death.

FIG. 4b: Growth of the BLOCKADE cells (containing an episomal version of GFP) on minimum medium with 2% galactose containing 2.5 and 1.25 μM of compound BLK200721. The WT cells were grown on minimum medium with 2% galactose containing 2.5 μM of compound BLK200721.

FIG. 4c: Representative example of fluorescence production when cells are submitted to increasing concentrations of inhibitor compound BLK200775. Higher concentrations than 15 μM result in cell death.

FIG. 4d: Growth of the BLOCKADE cells (containing an episomal version of GFP) on minimum medium with 2% galactose containing 15, 10 and 5 μM of compound BLK200775. The WT cells were grown on minimum medium with 2% galactose containing 15 μM of compound BLK200775. Measurement of GFP with 15 μM was carried after 130 h and not represented in the graph.

### EXAMPLES

The example relate to:
a) genetically modified yeast cell, deleted for BNA2 and NPT1 genes and carrying the BNA2 promoter fused to a fluorescent reporter protein integrated into its genome and the human IDO gene, grown in selective media conditions;
b) a screening model system based on the presence/absence of galactose, glucose, tryptophan in different combinations in the presence/absence of IDO activity modulators, and
c) a model for determining the less toxic but more potent dosage of each potential inhibitor.

The construct described in this example therefore consists in the co-deletion of the BNA2 and NPT1 genes, with introduction of the human IDO gene. The construct and cell was built with the following steps:
a. providing a yeast cell where the entire BNA2 coding sequence was popped out from the genome by the introduction of a reporter gene directly downstream the BNA2 promoter. In the present construct, the reporter gene consists in the EGFP gene, but any other suitable reporter gene (XFP, β-galactosidase, luciferase or other reporter genes known of the skilled artisan) can be used. This gives a stable expression of the reporter gene.
b. controlling the expression of the IDO gene under a specific/constitutive/inducible promoter in the previously said cell, by growing the said cell on selective medium. In one example, the utilisation of an expression vector containing the GAL1 promoter induces the expression of said IDO when the cells are grown on galactose. The yeast expression vector containing the IDO gene is then used as a rescue plasmid for later NPT1 deletion.
c. deletion of NPT1 by two-step gene replacement with the said cells grown on galactose to induce IDO and thus, to maintain the kynurenine pathway active and keep the host cells alive. The present invention presents therefore a completely artificial kynurenine pathway, tightly controllable by the skilled user. In another embodiment to obtain the same construct, the double deletion can be performed by first the deletion of NPT1 and finally the deletion of BNA2.
d. identification of inhibitors of said IDO by contacting said host cells with a compound under suitable conditions, such that the inhibition of the IDO activity leads to cell death. In one embodiment, the minimal concentration of said inhibitor that prevents cell growth is then determined. As used herein, “contacting” the yeast cell with a compound refers to exposing, incubating, touching, associating, making accessible the yeast cell to the compound.
e. identification of the minimum inhibition concentration of the said compounds by contacting said host cells under suitable conditions such that the inhibition of the IDO activity leads to fluorescence emission. In an additional embodiment, induction of the reporter gene will then support the specific action of the said inhibitor on the IDO enzyme.

One aspect of the invention allows the detection of modification in the NAD⁺ levels inside the cell by formation of EGFP, through the control of the BNA2 promoter. Other aspects of the invention relate to any other gene involved in NAD⁺ synthesis, in particular to genes of the kynurenine pathway. In another embodiment, the invention relates to human kynurenine 3-monooxygenase gene (K3M) with cloning of the human K3M gene in the inducible vector and deletion of the corresponding gene in yeast (Table 2). Even though IDO is the rate limiting step in the kynurenine pathway, the invention includes not only screening of IDO inhibitors, but also to other enzymes, tightly linked to the kynurenine pathway (Table 2) and to the control of NAD⁺/NADH levels in the cell.

TABLE 2
Mammalian enzymes of the tryptophan to NAD⁺ pathway and their
yeast equivalents. The mammalian enzymes are most suitable for
determining potential therapeutic agents.

| | Equivalent yeast |
|---|---|
| Mammalian Enzyme | gene |
| Indoleamine 2,3-dioxygenase | BNA2 |
| Tryptophan 2,3.dioxygenase | BNA2 |
| Formamidase | BNA3 |
| Kynurenine amino-transferase | ARO8/ARO9 |
| Kynureninase | BNA5 |
| Kynurenine 3-hydroxylase | BNA4 |
| Kynurenine 3-monooxydase | BNA4 |
| 3-hydroxyanthranilic acid dioxygenase | BNA1 |
| Quinolinate phosphoribosyl transferase | BNA6 |
| Nicotinate phosphoribosyl transferase | NPT1 |
| Nicotinamide/nicotinic acid mononucleotide | NMA1 |
| adenylyltransferase | |
| Nicotinamide/nicotinic acid mononucleotide | NMA2 |
| adenylyltransferase | |
| Glutamine-dependent NAD synthase | QNS1 |
| NAD-dependent histone deacetylase | SIR2 |
| Nicotinamidase | PNC1 |

The platform here proposed can therefore be used to screen inhibitors/modulators of any enzymes associated with tryptophan metabolism and NAD⁺/NADH levels. The invention, because of the exploitation of the tryptophan to NAD⁺ pathway, including the BNA2 and other promoter properties as a NAD⁺ sensor/probe, can therefore be adapted to any kind of cellular mechanisms involving modification of NAD⁺ levels (such as aging, lifespan extension, cancer and degeneration diseases).

### Methods of the Examples

### Example 1

The following methods describe the construction of suitable host cells and other molecular biological reagents necessary for the development and the use of the present invention.

### Yeast Strains and Transformation

In this study, we used the yeast strain Y00000 (MA Ta; his3Δ1; leu2Δ0; met15Δ0; ura3Δ0) as host cell for all the constructs and for chromosomal DNA isolation. Transformation of yeast cells was performed accordingly to the LiAc method (Gietz, D., A. St. Jean, R. A. Woods, and R. H. Schiestl. 1992, Nucleic Acids Res. 20:1425).

Integration of the reporter gene into the BNA2 coding sequence by homologous recombination to achieve stable reporter expression under BNA2 promoter induction.

A 1700 bps fragment (PromBNA2) directly upstream BNA2 coding sequence was amplified by PCR (SEQ ID NO:1) and cloned into pMOSBlue vector. A 200 bps fragment (TermBNA2) directly downstream BNA2 coding sequence was amplified by PCR (SEQ ID NO:2) and cloned in pBlueScript KS. The fragment PromBNA2 was digested with EcoRI and BamHI, giving a 1050 bps insert corresponding to the 5′ upstream region of BNA2. The resulting insert was then cloned directly in front of the reporter coding sequence. In a first embodiment, the reporter gene is the EGFP gene, giving rise to the plasmid pEGFP_ PromBNA2. The pEGFP_PromBNA2 plasmid was then digested with AMlII, blunt-ended and again digested with HindIII. The resulting 2050 bps fragment was consequently cloned into the yeast episomal vector YEplac181, digested with HindIII and SmaI, resulting in the plasmid YEpPromBNA2-EGFP. The TermBNA2 fragment was extracted from pBlueScript by digestion with KpnI and SacI and then cloned into YEpPromBNA2-EGFP just after EGFP, giving the plasmid YEpΔbna2-EGFP.

The plasmid YEpΔbna2-EGFP was digested with PvuII, resulting in a fragment containing 500 bps of PromBNA2, the EGFP coding sequence and 200 bps TermBNA2. This fragment was then transformed in the yeast Y00000 in order to promote integration into the BNA2 locus, therefore replacing the endogenous BNA2 coding sequence with the EGFP coding sequence. This gave rise to the S. cerevisiae strain Y Δbna2:EGFP.

In a further preferred embodiment the reporter gene herein referred to can be any other suitable reporter gene (XFP, β-galactosidase, luciferase or other reporter genes known of the skilled artisan).

Cloning of the IDO Gene in a Yeast Episomal Vector and Expression in Y Δbna2:EGFP S. cerevisiae

The IDO gene was obtained from the IMAGE consortium as a 1586 bps fragment cloned in the vector pCMV-SPORT6. The pCMV-SPORT6 plasmid was digested with SmaI and XhoI to isolate the IDO fragment. The generated fragment was then cloned in the episomal yeast vector, directly downstream the GAL1 promoter. The resulting plasmid pBIOALVO-IDO was transformed in the strain Y Δbna2:EGFP, resulting in the strain YEGFP-IDO. Expression of the IDO was induced by growing the cell on 2% galactose. Presence of the active protein was verified by Western blotting and by measurement of IDO activity from cell extracts. Western blotting can easily be performed by people of the art and determination of IDO activity has already been described in the literature (Takikawa O., T. Kuroiwa, F. Yamazaki and R. Kido, 1998, J. Biol. Chem 263:2041-2048).

### Deletion of NPT1 in the Strain YEGFP-IDO

A 560 bps fragment directly upstream NPT1 coding sequence (PromNPT1) was amplified by PCR (SEQ ID NO:3). A 200 bps fragment directly downstream NPT1 coding sequence (TermNPT1) was also amplified by PCR (SEQ ID NO: 4). The 560 bps fragment PromNPT1 was digested with EcoRI and BamHI, leading to a 440 bps fragment, and then cloned in the integrative yeast vector YIplac211, giving rise to the plasmid YIpPromNPT1. The PCR product corresponding to TermNPT1 was then digested BamHI and XhoI and cloned in YIpPromNPT1, leading to the plasmid YIpΔnpt1. YIpΔnpt1 was linearised with MfeI (cutting inside PromNPT1 sequence) and transformed in the strain YEGFP-IDO. Transformants were plated on galactose to induce IDO expression and maintain the cell alive. NPT1 coding sequence was then popped out by selection on 5-FOA and mutants were confirmed by PCR and by lethality when the cells are plated on glucose (the IDO is not induced and the cell cannot survive without NAD⁺ synthesis). The strain YBLOCKADE was selected as the fastest growing on galactose.

### Screening Methods of the Invention

A method of performing the screen is to measure the fluorescence emission in 96-well microplates using a microplate reader, allowing the user high throughput screening of thousand candidate inhibitors. EGFP was excited at 488 nm and emitted fluorescence was measured at 507 nm. Cells were grown in 200 μl selective minimum medium (yeast nitrogen base without leucine) containing 20 g l⁻¹ galactose. Another method can be based on the measurement of the expression of any chosen reporter gene.

(a) Candidate substances:

The candidate compounds of any of the methods of the invention can be selected from chemical or biological libraries or natural product libraries. The candidate compound is any substance with a potential to reduce or alleviate completely the activity of IDO protein by competitive, uncompetitive, non-competitive or even unidentified mechanism of inhibition. Candidate substances include new tryptophan derivatives, indole derivatives or any unidentified compounds isolated from microorganisms, animals, plants or any other living organisms which are used to extract possible effective inhibitory agents.

(b) High throughput screening:

The robustness of the presented platform can be tested with already known IDO inhibitors such as 1-methyl-tryptophan (1MT), 7-methyl-tryptophan (7MT), and methyl-thiohydantoin-tryptophan (MTH-Tryp). Cells were grown in 96-well microplates, as previously described. Increasing concentrations of inhibitors are tested until getting a fluorescence signal. This gives us the minimal efficient dose of inhibitor. If cell death was observed, the concentration of inhibitor is decreased consequently.

TABLE 3
EGFP-based screening of IDO inhibitors. The first step is based on
life/death selection and then, various concentrations of inhibitor are
tested that allow growth and production of fluorescence.

| Glucose | Galactose | Inhibitor | Growth | Fluorescence |
|---|---|---|---|---|
| + | − | − | − | − |
| − | + | − | +++ | − |
| − | + | +++ | − | − |
| − | + | ++ | + | +++ |
| − | + | + | ++ | + |

+: very mild IDO inhibition/cell growth,
++: mild IDO inhibition/cell growth,
+++: strong IDO inhibition/cell growth,
−: no sugar/inhibitor/growth/fluorescence.

The design of this platform was made in such a way that the screening for inhibitors of the tryptophan to NAD⁺ pathway and/or modulators of NAD⁺/NADH levels causes a life or death phenotype in a first round and thereafter the dosage is determined by means of a signal increase proportional to the dose given. Being so, the embodiment described provides a detection and quantification method for the determination of IDO inhibition and inhibitor molecules. Methods to obtain genetically modified strains as well as to various different constructs of the platform are given. Also, all the details for the screening set up are also provided herein.

### Example 2

### Spotting Experiments

BLOCKADE cells (from the YBLOCKADE strain mentioned in Example 1) were routinely grown until they reach OD 1.5 at 30° C. in selective medium containing a mixture of 1.2% glucose and 0.8% galactose. The cells were then washed three times, resuspended in sterile water to a cell concentration of 10⁶ cells/ml. Six serial dilutions were spotted on selective medium supplemented with 2% galactose (FIG. 2, Panel A) or 2% glucose (FIG. 2, Panel B).

Induction of IDO by galactose promoted the growth of the BLOCKADE strains, whereas glucose repressed the induction of IDO and this inhibited growth of the BLOCKADE strains. The same type of experiment was repeated in liquid media and the results were the same. This confirms the dependency of the cells on IDO activity to grow and allows a primary life/death assay screen.

### Example 3

### Growth Inhibition with Small Molecule Compounds from Libraries

Cells were pre-grown on a mixture of 1.2% glucose and 0.8% galactose until reaching early exponential phase (OD=1-2). The cells were washed three times with water and then resuspended in fresh selective medium containing 2% galactose, in order to fully activate IDO expression. Cells were dispensed into 96-well plates and inhibitor compounds were added to the cell culture at concentrations of 10, 5, 2.5 and 1 μM. Growth was monitored during 3 days at 30° C. under agitation. Compounds were selected from diverse libraries.

IDO inhibitory compounds were identified as those that repressed the growth of the cells. Possible cytotoxic effects of the compounds were identified by growing the wild type strain in the same condition at a concentration of 10 μM. Compounds inhibiting both BLOCKADE cells and wild type strains were considered as cytotoxic and therefore not selected for further development. Compounds inhibiting only BLOCKADE cells, but not the wild type strains were considered as hits, potential IDO inhibitory compounds.

Inhibition by new compound BLK200735 is illustrated in FIG. 3a, inhibition by new compound BLK200736 is illustrated in FIG. 3b, inhibition by new compound BLK200721 is illustrated in FIG. 3c and inhibition by new compound BLK200775 is illustrated in FIG. 3d.

Growth inhibition of the cells by small molecule compounds demonstrates the robustness of the present invention. Sensitivity to different inhibitor concentrations further confirms its use as a high throughput screening platform for IDO inhibitors that can predict cytotoxicity effects of compounds very early in development.

FIG. 3e illustrates inhibition by compound BLK200769 (an indole derivative), which is a known IDO inhibitor. BLK200769 is as follows:

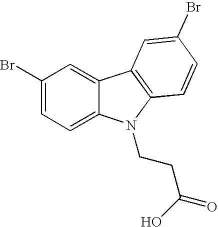

### Example 4

### Induction of Fluorescence Signal Relative to Inhibition of IDO

BLOCKADE cells used in this example were transformed with an episomal version of the plasmid carrying the GFP reporter gene under the control of the BNA2 promoter. This will ensure a higher fluorescence signal. Cells were pre-grown on a mixture of 1.2% glucose and 0.8% galactose until reaching early exponential phase (OD=1-2). The cells were washed three times with water and then resuspended in fresh selective medium containing 2% galactose, in order to fully activate IDO expression. Cells were dispensed into 96-well plates and previously identified inhibitor compounds were added to the cell culture at concentrations chosen in a way that the cells are only partially inhibited. Growth was monitored during 5 days at 30° C. under shaking.

The measurements of fluorescence from GFP were carried out using excitation and emission wavelengths at 485 nm and 520 nm, respectively. Fluorescence was normalized to the cell number (by normalizing the fluorescence signal to the absorbance signal). In order to compare the effect of the compound on the cells at the different concentrations, the time of entrance into the stationary phase was chosen as a reference point. At that point, the normalized fluorescence from the BLOCKADE cells cultivated without inhibitor was subtracted from the normalized fluorescence from the BLOCKADE cells exposed to different concentrations of inhibitor, resulting in a corrected fluorescence, directly representative of the inhibitor activity—the higher the corrected fluorescence, the higher the activity. This results from the fact that when IDO present in the cells is inhibited, the BNA2 promoter is not repressed, and therefore the GFP gene is expressed.

### Example 4a

The BLOCKADE cells (with an episomal version of GFP) were submitted to 0, 1.25 and 2.5 μM of compound BLK200721. As the concentration of inhibitor increases, IDO activity is decreased and the cell relies on a weaker IDO activity to survive. As NAD⁺ synthesis is limited, the BNA2 promoter is activated, resulting in higher GFP expression (see FIG. 4a). Growth of these cells on galactose containing media in the presence of BLK200721 was also measured and is illustrated in FIG. 4b.

### Example 4b

The BLOCKADE cells (with an episomal version of GFP) were submitted to 0, 5, and 15 μM of compound BLK200775. With increased concentrations of inhibitor, IDO activity is decreased and the cell relies on a weaker IDO activity to survive. As NAD⁺ synthesis is limited, the BNA2 promoter is activated, resulting in higher GFP expression (see FIG. 4c). Growth of these cells on galactose containing media in the presence of BLK200775 was also measured and is illustrated in FIG. 4d.

Examples 4a and 4b further demonstrate the use of the present invention not only to screen and select compounds with inhibitory activity on IDO, but also to determine the activity of the newly discovered compounds, without the need to purify the IDO protein and consequently use in vitro assays to measure the IDO protein, as is required in known procedures when assessing compound activity.

1. A cell suitable for screening a candidate agent as being an inhibitor of the metabolism of tryptophan to NAD⁺ and/or a modulator of NAD⁺ levels, which cell comprises functional genes of a pathway enabling the metabolism of tryptophan to NAD⁺ and wherein the cell includes a copy of an exogenous gene of said pathway, from the same or different species as the cell, which exogenous gene is under the control of an inducible or constitutive promoter and wherein any endogenous copy of the gene having the same function as the exogenous gene is a non-functioning gene.

2. A cell, as claimed in claim 1, wherein the copy of the exogenous gene of said pathway is outside of the genome of the cell.

3. A cell, as claimed in claim 1, which further comprises a reporter gene under the control of a promoter, which promoter is regulated, directly or indirectly, by the expression product of the exogenous gene.

4. A cell, as claimed in claim 3, wherein the promoter of the reporter gene is downregulated, directly or indirectly, by the expression product of the exogenous gene.

5. A cell, as claimed in claim 1, which is a eukaryotic cell.

6. A cell, as claimed in claim 5, which is a yeast cell, a human cell or a mouse cell.

7. A cell, as claimed in claim 1, which cell comprises the functional genes of a single pathway enabling the metabolism of tryptophan to NAD⁺.

8. A cell, as claimed in claim 1, wherein the exogenous gene is a human gene.

9. A cell, as claimed in claim 1, wherein the exogenous gene encodes any one of the following:
Indoleamine 2,3-dioxygenase, tryptophan 2,3-dioxygenase, formamidase, kynurenine amino-transferase, kynureninase, kynurenine 3-hydroxylase, kynurenine 3-monooxydase, 3-hydroxyanthranilic acid dioxygenase, quinolinate phosphoribosyl transferase, nicotinate phosphoriboxyl transferase, nicotinamide/nicotinic acid mononucleotide adenylyltransferase, nicotinamide/nicotinic acid mononucleotide adenylyltransferase, glutamine-dependent NAD synthase, NAD-dependent histone deacetylase, nicotinamidase.

10. A cell, as claimed in claim 9, wherein the exogenous gene encodes indoleamine 2,3-dioxygenase and wherein the promoter of the reporter gene is the promoter of the BNA2 gene.

11. A cell, as claimed in claim 1, wherein any gene enabling the metabolism of NA to NaNM is non-functioning.

12. A population of cells, as claimed in claim 1.

13. A method of screening a candidate agent for its ability to inhibit the metabolism of tryptophan to NAD⁺ and/or to modulate NAD⁺ levels, the method comprising contacting the candidate agent with a cell as claimed in claim 1, and determining the ability of the candidate agent to inhibit the metabolism of tryptophan to NAD⁺ and/or to modulate NAD⁺ levels.

14. A method of screening a candidate agent for its ability to inhibit the metabolism of tryptophan to NAD⁺ and/or to modulate NAD⁺ levels, the method comprising contacting the candidate agent with a cell, as claimed in claim 3, and determining the ability of the candidate agent to inhibit the metabolism of tryptophan to NAD⁺ and/or modulate NAD⁺ levels, wherein the ability of the candidate agent to inhibit the metabolism of tryptophan to NAD⁺ and/or to modulate NAD⁺ levels is determined by the level of expression of the reporter gene.

15. An agent which inhibits the metabolism of tryptophan to NAD⁺ and/or modulates NAD⁺ levels, obtained by a method as claimed in claim 13.

16. A method of treating a patient who suffers or is predisposed to suffer from a disease which requires inhibition of the metabolism of tryptophan to NAD⁺ and/or modulation of NAD⁺ levels for amelioration, the method comprising screening a candidate agent as claimed in claim 13 to identify an agent which inhibits the metabolism of tryptophan to NAD⁺ and/or modulates NAD⁺ levels and administering the agent to the patient.

17. A method of treating a patient who suffers or is predisposed to suffer from a disease which requires inhibition of the metabolism of tryptophan to NAD⁺ and/or modulation of NAD⁺ levels for amelioration with a therapeutic agent, wherein the therapeutic agent is obtained from a method as claimed in claim 13.

18. A method of screening a candidate agent for its ability to inhibit the metabolism of tryptophan to NAD⁺ and/or to modulate NAD⁺ levels, the method comprising contacting the candidate agent with a population of cells as claimed in claim 12, and determining the ability of the candidate agent to inhibit the metabolism of tryptophan to NAD⁺ and/or to modulate NAD⁺ levels.

19. An agent which inhibits the metabolism of tryptophan to NAD⁺ and/or modulates NAD⁺ levels, obtained by a method as claimed in claim 14.

20. A method of treating a patient who suffers or is predisposed to suffer from a disease which requires inhibition of the metabolism of tryptophan to NAD⁺ and/or modulation of NAD⁺ levels for amelioration, the method comprising screening a candidate agent as claimed in claim 14 to identify an agent which inhibits the metabolism of tryptophan to NAD⁺ and/or modulates NAD⁺ levels and administering the agent to the patient.

21. A method of treating a patient who suffers or is predisposed to suffer from a disease which requires inhibition of the metabolism of tryptophan to NAD⁺ and/or modulation of NAD⁺ levels for amelioration with a therapeutic agent, wherein the therapeutic agent is obtained from a method as claimed in or claim 14.
